(19) United States
(12) Patent Application Publication
(10) Pub. No.: US 2020/0078489 A1
(43) Pub. Date: Mar. 12, 2020

(54) PANCREATIC FISTULA OCCLUSION

(71) Applicant: 3-D Matrix, Ltd., Tokyo (JP)

(72) Inventor: Satoru Kobayashi, Chigasaki (JP)

(21) Appl. No.: 16/085,804

(22) PCT Filed: Mar. 17, 2017
(86) PCT No.: PCT/IB2017/000366

§ 371 Date: Sep. 17, 2018

Related U.S. Application Data
(60) Provisional application No. 62/310,124, filed on Mar. 18, 2016.

(51) Int. Cl. A61L 24/10; A61L 24/00

(52) U.S. Cl. CPC A61L 24/10; A61L 24/0031; A61L 24/0015

(57) ABSTRACT

Methods and materials for occluding a pancreatic fistula are described herein. One method for occluding a pancreatic fistula includes administering an effective amount of a self-assembling peptide solution to a pancreatic fistula, where the self-assembling peptide is between about 7 amino acids and 32 amino acids in length and the self-assembling peptide solution forms a hydrogel under physiological conditions, thereby occluding the pancreatic fistula.

### FIELD OF THE INVENTION

This disclosure generally relates to materials and methods that may be used in medical, research, and industrial applications. More particularly, this disclosure relates to materials and methods that may be used for pancreatic fistula occlusion.

### BACKGROUND

The pancreas is a glandular organ in the digestive system and endocrine system that produces several important hormones (e.g., insulin, glucagon, somatostatin, and pancreatic polymeptide) and pancreatic juice containing digestive enzymes that help to breakdown carbohydrates, proteins, and lipids in the chyme.

A pancreatic fistula is an abnormal communication between the pancreas and other organs due to leakage of pancreatic secretions from damaged pancreatic ducts. Pancreatic fistula may occur during various digestive system surgeries. Blood may sometimes leak with the fluid. The pancreatic fistula may be observed post-operatively during examination. Pancreatic fistula may cause severe complications such as organ bleeding, particularly with digestive action by pancreas fluids.

Existing therapies, such as expanded fibrin glue, may have limited efficacy. Accordingly, there remains a need for improved treatments for pancreatic fistulas.

### SUMMARY

The invention is based, at least in part, upon the discovery that certain amphiphilic peptide solution can be surprisingly and advantageously used to treat pancreatic fistulas.

In various aspects, the invention provides a method for occluding a pancreatic fistula, the method comprising administering an effective amount of a self-assembling peptide solution to a pancreatic fistula, wherein the self-assembling peptide is between about 7 amino acids and 32 amino acids in length and the self-assembling peptide solution forms a hydrogel under physiological conditions, thereby occluding pancreatic fistula.

In various aspects, the invention provides a use of an effective amount of a self-assembling peptide solution for occluding a pancreatic fistula, wherein the self-assembling peptide is between about 7 amino acids and 32 amino acids in length and the self-assembling peptide solution forms a hydrogel under physiological conditions, thereby occluding pancreatic fistula.

In various aspects, the invention provides a method for pancreatic fistula occlusion comprising introducing a delivery device to a target area associated with a pancreatic fistula, positioning an end of the delivery device in the target area at which occlusion is desired, administering through the delivery device a solution comprising a self-assembling peptide comprising between about 7 amino acids and 32 amino acids in an effective amount and in an effective concentration to the target area to form a hydrogel under physiological conditions of the target area to promote occlusion; and removing the delivery device from the target area.

In various aspects, the invention provides a composition comprising a self-assembling peptide comprising between about 7 amino acids and 32 amino acids in an effective amount and in an effective concentration for use in forming a hydrogel under physiological conditions to promote pancreatic fistula occlusion. Pancreatic fistula occlusion can be plugging, preventing passage, closing, obstructing, enclosing, or closing off of an opening or a hole through which pancreatic fluid is leaking.

In various aspects, the invention provides a kit for promoting pancreatic fistula occlusion comprising a self-assembling peptide comprising between about 7 amino acids and about 32 amino acids in an effective amount to form a hydrogel under physiological conditions to promote occlusion, and instructions for administering the self-assembling peptide to a target area associated with a pancreatic fistula.

In various aspects, the invention provides a method of facilitating pancreatic fistula occlusion, comprising providing a solution comprising a self-assembling peptide comprising between about 7 amino acids to about 32 amino acids in an effective amount and in an effective concentration to form a hydrogel in a target area associated with a pancreatic fistula under physiological conditions to promote occlusion; and providing instructions for administering the solution to the target area through introduction of the solution through a delivery device positioned in the target area.

In various aspects, the invention provides a macroscopic scaffold consisting essentially of a plurality of self-assembling peptides, each of the self-assembling peptides comprising between about 7 amino acids and about 32 amino acids in an effective amount that is capable of being positioned within a target area associated with a pancreatic fistula to promote occlusion.

As will be understood by those skilled in the art, any of the aspects above can be combined with any one or more of the features below.

In various embodiments, the self-assembling peptide comprises about 12 to about 16 amino acids that alternate between hydrophobic and a hydrophilic amino acids.

In various embodiments, the self-assembling peptide comprises a sequence selected from RADA, IEIK, TTTT, ATAT, TVTV, ASAS, SSSS, VVVTTTT, and a combination thereof.

In various embodiments, the self-assembling peptide comprises a sequence selected from (RADA)₄, (IEIK)₃I, and (KLDL)₃.

In various embodiments, the self-assembling peptide is about 0.1 to about 10 w/v % of the solution or about 0.1 to about 3.5 w/v % of the solution.

In various embodiments, the self-assembling peptide is about 1, about 2.5, or about 3 w/v % of the solution.

In various embodiments, the effective amount is approximately 0.1 mL per 1 cm² to approximately 5 mL per 1 cm² of target area.

In various embodiments, the effective amount is approximately 1 mL per 1 cm² of target area.

In various embodiments, the hydrogel is formed before administering the self-assembling peptide solution to the pancreatic fistula.

In various embodiments, the hydrogel is formed after administering the self-assembling peptide solution to the pancreatic fistula.

In various embodiments, the solution further comprises a biologically active agent.

In various embodiments, the solution is substantially free of cells and/or drugs.

In various embodiments, the self-assembling peptide solution is administered in vivo.

In various embodiments, the pancreatic fistula is a human pancreatic fistula.

In various embodiments, the self-assembling peptide comprises between about 12 to about 16 amino acids that alternate between a hydrophobic amino acid and a hydrophilic amino acid.

In various embodiments, the solution is an aqueous solution and wherein a concentration of the peptide in the aqueous solution is about 0.1 weight per volume (w/v) percent to about 3 w/v percent.

In various embodiments, the method further comprises visualizing the target area prior to introducing and/or subsequent to removing the delivery device from the target area.

In various embodiments, the method further comprises monitoring the target area after removing the delivery device.

In various embodiments, the effective amount is approximately 1 mL per 1 cm² of target area.

In various embodiments, the method further comprises preparing the solution comprising the self-assembling peptide.

In various embodiments, the self-assembling peptide is selected from the group consisting of (RADA)₄, (IEIK)₃I, and (KLDL)₃.

In various embodiments, the solution further comprises a biologically active agent.

In various embodiments, the target site relates to a digestive system surgery.

In various embodiments, the hydrogel occludes pancreatic fluid leaking from the pancreas or accelerates regeneration of the pancreas.

In various embodiments, the solution is substantially free of cells and/or drugs.

In various embodiments, the concentration effective to promote occlusion comprises a self-assembling peptide concentration in a range of about 0.1 weight per volume (w/v) percent to about 3 w/v percent.

In various embodiments, the composition is substantially free of cells and/or drugs.

In various embodiments, the composition is used for promoting pancreatic fistula occlusion.

These and other advantages of the present technology will be apparent when reference is made to the following description.

### DETAILED DESCRIPTION

The invention is based, at least in part, upon the discovery that certain amphiphilic peptide solution can be surprisingly and advantageously used to treat pancreatic fistulas.

In various aspects and embodiments, the invention provides methods and materials for occluding a pancreatic fistula. The method includes administering an effective amount of a self-assembling peptide solution to a pancreatic fistula, where the self-assembling peptide is between about 7 amino acids and 32 amino acids in length and the self-assembling peptide solution forms a hydrogel under physiological conditions.

In accordance with one or more embodiments, self-assembling peptide hydrogels may facilitate pancreatic fistula occlusion. Their efficacy as a hemostat has been demonstrated. The hydrogels may exhibit efficacy for pancreatic fistula occlusion that is equal to or greater than that of fibrin glue. Presence of blood and pH level may be factors for efficacy of the hydrogels for pancreatic occlusion. The self-organized peptides may occlude pancreatic fluid leaking from the pancreas. The self-organized peptides may accelerate regeneration of the pancreas. Occluding of pancreatic fluid leak can be plugging, preventing passage, closing, obstructing, enclosing, or closing off of an opening or a hole through which pancreatic fluid is leaking.

The methods and materials may comprise occlusion of a pancreatic fistula. The occlusion may be partial or complete. The materials and methods may include administration, application, or injection of a self-assembling peptide, or a solution comprising a self-assembling peptide, or a composition comprising a self-assembling peptide, to a predetermined or desired target area.

Through administration of the solution comprising the self-assembling peptide, a hydrogel barrier may be formed. The hydrogel barrier may be formed in the target area to promote occlusion. The self-organizing or self-assembling peptides of the present disclosure may include application of the self-organizing or self-assembling peptides to a predetermined or desired target. The self-organizing or self-assembling peptide may be applied or introduced to a target site in the form of a peptide solution, hydrogel, membrane or other form. A target site may be a predetermined area of a subject that requires a particular treatment. In some embodiments, the target site may relate to a digestive system surgery. In some embodiments, the target site may relate to a surgical site or the site of a fistula. For example, a surgical site may be a site where surgery was performed or a fistula has developed.

During self-organization or self-assembly, the peptide may form nanofibers. The self-organization or self-assembly may cause gelling of the peptide in solution. The gelling may provide or form a hydrogel. The peptide may form a beta-sheet spontaneously in the solution under the physiological pH level. The peptide may form a beta-sheet spontaneously in the solution under physiological conditions and/or in the presence of a cation.

Various features of the invention are discussed, in turn, below.

As used herein, the term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

The term “self-assembling peptide” may refer to a peptide comprising a self-assembling motif. Self-assembling peptides are peptides that are capable of self-assembly into structures including but not limited to, macroscopic membranes or nanostructures. For example, the self-assembling peptide may exhibit a beta-sheet structure in aqueous solution in the presence of specific conditions to induce the beta-sheet structure. These specific conditions may include adjusting the pH of a self-assembling peptide solution. The adjustment may be an increase or a decrease in the pH of the self-assembling peptide solution. The increase in pH may be an increase in pH to a physiological pH. The specific conditions may also include adding a cation, such as a monovalent cation, to a self-assembling peptide solution. The specific conditions may include conditions related to the pancreas. The self-assembling peptides may be referred to as or be a part of a composition, peptide solution, peptide powder, hydrogel, or scaffold. The self-assembling peptide may be administered to a target area in the form of a peptide solution, composition, hydrogel, membrane, scaffold or other form.

The term “hydrogel” may refer to a material that is comprised of a polymer and a high percentage of water, for example, at least 90% water.

The self-assembling peptide may be an amphiphilic self-assembling peptide. By “amphiphilic” it is meant that the peptide comprises hydrophobic portions and hydrophilic portions. In some embodiments, an amphiphilic peptide may comprise, consist essentially of, or consist of alternating hydrophobic amino acids and hydrophilic amino acids. By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid. In certain embodiments, the peptide may comprise a first portion that is amphiphilic and a second portion that is not amphiphilic.

The self-assembling peptide, also referred to herein as “peptide” or “self-assembling oligopeptides,” may be administered to the pre-determined or desired target area in the form of a self-assembling peptide solution, composition, hydrogel, membrane, scaffold or other form. The hydrogel may also be referred to as a membrane or scaffold throughout this disclosure.

The pre-determined or desired target area may be at or near the location of a surgery or the site of a pancreatic fistula. The pre-determined or desired target area may be established based on the site of or other area that may have undergone a surgical procedure, or an unintentional or intentional trauma.

The self-assembling peptide solution may be an aqueous self-assembling peptide solution. The self-assembling peptide may be administered, applied, or injected in a solution that is substantially cell-free, or free of cells. In certain embodiments, the self-assembling peptide may be administered, applied, or injected in a solution that is cell-free or free of cells.

The self-assembling peptide may also be administered, applied, or injected in a solution that is substantially drug-free or free of drugs. In certain embodiments, the self-assembling peptide may be administered, applied, or injected in a solution that is drug-free or free of drugs. In certain other embodiments, the self-assembling peptide may be administered, applied, or injected in a solution that is substantially cell-free and substantially drug-free. In still further certain other embodiments, the self-assembling peptide may be administered, applied, or injected in a solution that is cell-free and drug free.

The self-assembling peptide solution may comprise, consist of, or consist essentially of the self-assembling peptide. The self-assembling peptide may be in a modified or unmodified form. By modified, it is meant that the self-assembling peptide may have one or more domains that comprise one or more amino acids that, when provided in solution by itself, would not self-assemble. By unmodified, it is meant that the self-assembling peptide may not have any other domains other than those that provide for self-assembly of the peptide. That is, an unmodified peptide consists of alternating hydrophobic and hydrophilic amino acids that may self-assemble into a beta-sheet, or a macroscopic structure, such as a hydrogel.

The self-assembling peptide can be at least about 7 amino acids, between about 7 and 32 amino acids, or between about 12 and 16 amino acids. Other peptides that do not comprise, consist of, or consist essentially of at least about 7 amino acids may be contemplated by this disclosure. The self-assembling peptides may be composed of about 6 to about 200 amino acid residues. In certain embodiments, about 8 to about 32 residues may be used in the self-assembling peptides, while in other embodiments self-assembling peptides may have about 7 to about 17 residues. In certain other examples, the self-assembling peptides may be peptides of at least 8 amino acids, at least about 12 amino acids, or at least about 16 amino acids.

The materials and methods may comprise administering a self-assembling peptide to a predetermined or desired target. The peptide may be administered as a hydrogel or form a hydrogel upon administration. A hydrogel is a term that may refer to a colloidal gel that is dispersed in water. The hydrogel may also be referred to as a membrane or scaffold throughout this disclosure. The systems and methods may also comprise applying a self-assembling peptide to a predetermined or desired target as a solution such as an aqueous peptide solution.

The term “administering,” is intended to include, but is not limited to, applying, introducing or injecting the self-assembling peptide, in one or more of various forms including, but not limited to, by itself, by way of solution, such as an aqueous solution, or by way of a composition, hydrogel, or scaffold, with or without additional components.

The method may comprise introducing a delivery device at or near a predetermined or desired target area of a subject. The method may comprise introducing a delivery device comprising at least one of a syringe, tube, pipette, catheter, catheter syringe, or other needle-based device to the predetermined or desired target area of a subject. The self-assembling peptide may be administered by way of a syringe, tube, pipette, catheter, catheter syringe, or other needle-based device to the predetermined or desired target area of a subject. The gauge of the syringe needle may be selected to provide an adequate flow of a composition, a solution, a hydrogel, or a liquid from the syringe to the target area. This may be based in some embodiments on at least one of the amount of self-assembling peptide in a composition, peptide solution, or a hydrogel being administered, the concentration of the peptide solution, in the composition, or the hydrogel, and the viscosity of the peptide solution, composition, or hydrogel.

The delivery device may be a conventional device or designed to accomplish at least one of to reach a specific target area, achieve a specific dosing regime, deliver a specific target volume, amount, or concentration, and deliver accurately to a target area.

The method of occluding a pancreatic fistula may comprise introducing a delivery device into the subject and positioning an end of the delivery device in a predetermined or target area, such as a portion of a surgical site or a pancreatic fistula. The self-assembling peptide may be administered by way of a delivery device to the target area in which at least is desired. The use of a delivery device may provide a more selective administration of the peptide to provide for a more accurate delivery to the target area. Selective administration of the peptide may allow for enhanced and more targeted delivery of the peptide solution, composition, or hydrogel such that is successful and positioned in the desired location in an accurate manner. The selective administration may provide enhanced, targeted delivery that markedly improves the positioning and effectiveness of the treatment over use of another delivery device. Delivery devices that may be used in the systems, methods, and kits of the disclosure may include a syringe, tube, needle, pipette, syringe catheter, other needle-based device, or catheter.

Use of a delivery device, such as a catheter, may include use of accompanying devices, such as a guidewire used to guide the catheter into position, or an endoscope that may allow proper placement of the catheter and visualization of the target area, and/or the path to the target area. The endoscope may be a tube that may comprise at least one of a light and a camera or other visualization device to allow images of the subject's body to be viewed. The guidewire or endoscope may be introduced into the subject, for example, by way of an incision in the skin. The endoscope may be introduced to the target area prior to introducing the delivery device to the target area.

The use of the delivery device, such as a syringe, tube, needle, pipette, syringe catheter, other needle-based device, catheter, or endoscope may require determining the diameter or size of the opening in which there is a target area, such that at least a portion of the syringe, tube, needle, pipette, syringe catheter, other needle-type device, catheter, or endoscope may enter the opening to administer the peptide, peptide solution, composition, or hydrogel to the target area.

In certain embodiments, the hydrogel may be formed in vitro and administered to the desired location in vivo. In certain examples, this location may be the target area. In other examples, this location may be upstream, downstream of the area, or substantially near the area. It may be desired to allow a migration of the hydrogel to the area in which it is desired to. Alternatively, another procedure may position the hydrogel in the area in which it is desired. The desired location or target area may be at least a portion of an area in which it is desired to provide or promote an occlusion at or near a pancreatic fistula in a subject.

In certain aspects of the disclosure, the hydrogel may be formed in vivo. A solution comprising the self-assembling peptide, such as an aqueous solution, may be inserted to an in vivo location or area of a subject to promote or provide an occlusion at or near a pancreatic fistula in a subject. In certain examples, the hydrogel may be formed in vivo at one location, and allowed to migrate to the area in which it is desired to promote or provide an occlusion at or near a pancreatic fistula in a subject. Alternatively, another procedure may place the hydrogel in the area in which it is desired to promote or provide an occlusion at or near a pancreatic fistula in a subject. The peptides of the present disclosure may be in the form of a powder, a solution, a gel, or the like. Since the self-assembling peptide gels in response to changes in solution pH and salt concentration, it can be distributed as a liquid that gels upon contact with a subject during application or administration.

In certain environments, the peptide solution may be a weak hydrogel and, as a result, it may be administered by way of a delivery device as described herein.

In accordance with some embodiments, the self-assembling peptides may be amphiphilic, alternating between hydrophobic amino acids and hydrophilic amino acids.

In accordance with one or more embodiments, a subject may be evaluated to determine a need to promote or provide an occlusion at or near a pancreatic fistula in a subject. Once the evaluation has been completed, a peptide solution to administer to the subject may be prepared.

In some embodiments, a biologically active agent may be used with the materials and methods of the present disclosure. A biologically active agent may comprise a compound, including a peptide, DNA sequence, chemical compound, or inorganic or organic compound that may impart some activity, regulation, modulation, or adjustment of a condition or other activity in a subject or in a laboratory setting. The biologically active agent may interact with another component to provide such activity. The biologically active agent may be referred to as a drug in accordance with some embodiments herein. In certain embodiments, one or more biologically active agents may be gradually released to the outside of the peptide system. For example, the one or more biologically active agents may be gradually released from the hydrogel. Both in vitro and in vivo testing has demonstrated this gradual release of a biologically active agent. The biologically active agent may be added to the peptide solution prior to administering to a subject, or may be administered separately from the solution to the subject. The one or more biologically active agents may be encapsulated within the system, for example, they may be encapsulated in the hydrogel, solution, or nanofibers.

The self-assembling peptides may exhibit a beta-sheet structure in aqueous solution in the presence of physiological pH and/or a cation, such as a monovalent cation, or other conditions applicable to a surgical site or at or near the site of a pancreatic fistula.

The peptides may be generally stable in aqueous solutions and self-assemble into large, macroscopic structures, scaffolds, or matrices when exposed to physiological conditions, physiological pH, or physiological levels of salt. Once the hydrogel is formed it may not decompose, or may decompose or biodegrade after a period of time. The rate of decomposition may be based at least in part on at least one of the amino acid sequence and conditions of its surroundings.

By “macroscopic” it is meant as having dimensions large enough to be visible under magnification of 10-fold or less. In preferred embodiments, a macroscopic structure is visible to the naked eye. A macroscopic structure may be transparent and may be two-dimensional, or three-dimensional. Typically each dimension is at least 10 μm, in size. In certain embodiments, at least two dimensions are at least 100 μm, or at least 1000 μm in size. Frequently at least two dimensions are at least 1-10 mm in size, 10-100 mm in size, or more. In certain embodiments, the size of the filaments may be about 10 nanometers (nm) to about 20 nm. The interfilament distance may be about 50 nm to about 80 nm. The self-assembling peptides of the present disclosure may have a length of about 5 nm. The self-assembling peptides of the present disclosure may have a nanofiber diameter in a range of about 10 nm to about 20 nm and an average pore size is in a range of about 5 nm to about 200 nm. In certain embodiments, the nanofiber diameter, the pore size, and the nanofiber density may be controlled by at least one of the concentration of peptide solution used and the amount of peptide solution used, such as the volume of peptide solution. As such, at least one of a specific concentration of peptide in solution and a specific amount of peptide solution to provide at least one of a desired nanofiber diameter, pore size, and density to adequately provide for an occlusion may be selected.

“Physiological conditions” may occur in nature for a particular organism, cell system, or subject which may be in contrast to artificial laboratory conditions. The conditions may comprise one or more properties such as one or more particular properties or one or more ranges of properties. For example, the physiological conditions may include a temperature or range of temperatures, a pH or range of pH's, a pressure or range of pressures, and one or more concentrations of particular compounds, salts, and other components. For example, in some examples, the physiological conditions may include a temperature in a range of about 20 to about 40 degrees Celsius. In some examples, the atmospheric pressure may be about 1 atm. The pH may be in the range of a physiological pH. For example, the pH may be in a range of about 6 to about 8. The physiological conditions may include cations such as monovalent metal cations that may induce membrane or hydrogel formation. These may include sodium chloride (NaCl). The physiological conditions may also include a glucose concentration, sucrose concentration, or other sugar concentration, of between about 1 mM and about 20 mM.

The peptides may also be complementary and structurally compatible. Complementary refers to the ability of the peptides to interact through ionized pairs and/or hydrogen bonds which form between their hydrophilic side-chains, and structurally compatible refers to the ability of complementary peptides to maintain a constant distance between their peptide backbones. Peptides having these properties participate in intermolecular interactions which result in the formation and stabilization of beta-sheets at the secondary structure level and interwoven filaments at the tertiary structure level.

Both homogeneous and heterogeneous mixtures of peptides characterized by the above-mentioned properties may form stable macroscopic membranes, filaments, and hydrogels. Peptides which are self-complementary and self-compatible may form membranes, filaments, and hydrogels in a homogeneous mixture. Heterogeneous peptides, including those which cannot form membranes, filaments, and hydrogels in homogeneous solutions, which are complementary and/or structurally compatible with each other may also self-assemble into macroscopic membranes, filaments, and hydrogels.

The membranes, filaments, and hydrogels may be non-cytotoxic. The hydrogels of the present disclosure may be digested and metabolized in a subject. The hydrogels may be biodegraded in 30 days or less. They have a simple composition, are permeable, and are easy and relatively inexpensive to produce in large quantities. The membranes and filaments, hydrogels or scaffolds may also be produced and stored in a sterile condition. The optimal lengths for membrane formation may vary with at least one of the amino acid composition, solution conditions, and conditions at the target site.

The amino acids of the self-assembling or amphiphilic peptides may be selected from d-amino acids, 1-amino acids, or combinations thereof. The hydrophobic amino acids may include Ala, Val, Ile, Met, Phe, Tyr, Trp, Ser, Thr and Gly. The hydrophilic amino acids may be basic amino acids, for example, Lys, Arg, His, Orn; acidic amino acids, for example, Glu, Asp; or amino acids which form hydrogen bonds, for example, Asn, Gln. Acidic and basic amino acids may be clustered on a peptide. The carboxyl and amino groups of the terminal residues may be protected or not protected. Membranes or hydrogels may be formed in a homogeneous mixture of self-complementary and self-compatible peptides or in a heterogeneous mixture of peptides which are complementary and structurally compatible to each other. Peptides fitting the above criteria may self-assemble into macroscopic membranes under suitable conditions, described herein.

The peptide may comprise or consist essentially of a sequence selected from the group consisting of: RADA, IEIK, TTTT, ATAT, TVTV, ASAS, SSSS, VVVTTTT, and combinations thereof. Other peptide sequences are contemplated and are within the scope of this disclosure. In certain embodiments, the peptide may comprise or consist essentially of a repeated sequence of arginine, alanine, and aspartic acid.

The peptides of the present disclosure may include peptides having the repeating sequence of arginine, alanine, aspartic acid and alanine (Arg-Ala-Asp-Ala (RADA)), and such peptide sequences may be represented by (RADA)ₚ, wherein p=2-50.

Other peptide sequences may be represented by self-assembling peptides having the repeating sequence of isoleucine, glutamic acid, isoleucine and lysine (Ile-Glu-Ile-Lys (IEIK)), and such peptide sequences are represented by (IEIK)ₚ, wherein p=2-50. Other peptide sequences may be represented by self-assembling peptides having the repeating sequence of isoleucine, glutamic acid, isoleucine and lysine (Ile-Glu-Ile-Lys (IEIK)), and such peptide sequences are represented by (IEIK)ₚI, wherein p=2-50.

Other peptide sequences may be represented by self-assembling peptides having the repeating sequence of lysine, leucine, aspartic acid, and leucine (Lys-Leu-Asp-Leu (KLDL)), and such peptide sequences are represented by (KLDL)ₚ, wherein p=2-50. Other peptide sequences may be represented by self-assembling peptides having the repeating sequence of lysine, leucine, and aspartic acid (Lys-Leu-Asp (KLD)), and such peptide sequences are represented by (KLD)ₚ, wherein p=2-50. As specific examples of self-assembling peptides according to the invention there may be a self-assembling peptide RADA16 having the sequence Arg-Ala-Asp-Ala-Arg-Ala-Asp-Ala- Arg-Ala-Asp-Ala-Arg-Ala-Asp-Ala (RADA)4, a self-assembling peptide IEIK13 having the sequence Ile-Glu-Ile-Lys-Ile-Glu-Ile-Lys-Ile-Glu-Ile-Lys-Ile (IEIK)₃I, a self-assembling peptide IEIK17 having the sequence Ile-Glu-Ile-Lys-Ile-Glu-Ile-Lys-Ile-Glu-Ile-Lys-Ile-Glu-Ile-Lys-Ile (IEIK)₄I or a self-assembling peptide KLDL12 having the sequence Lys-Leu-Asp-Leu-Lys-Leu-Asp-Leu-Lys-Leu-Asp-Leu (KLDL)₃.

The criteria of amphiphilic sequence, length, complementarity and structural compatibility apply to heterogeneous mixtures of peptides. For example, two different peptides may be used to form the membranes: peptide A, Val-Arg-Val-Arg-Val-Asp-Val-Asp-Val-Arg-Val-Arg-Val-Asp-Val-Asp (VRVRVDVDVRVRVDVD) has Arg and Asp as the hydrophilic residues and peptide B, Ala-Asp-Ala-Asp-Ala-Lys-Ala-Lys-Ala-Asp-Ala-Asp-Ala-Lys-Ala-Lys ADADAKAKADADAKAK, has Lys and Asp. Peptides A and B are complementary; the Arg on A can form an ionized pair with the Asp on B and the Asp on A can form an ionized pair with the Lys on B. Thus, in a heterogeneous mixture of peptides A and B, membranes would likely form, but they would be homogeneously composed of either peptide A or B.

Membranes and hydrogels can also be formed of heterogeneous mixtures of peptides, each of which alone would not form membranes, if they are complementary and structurally compatible to each other. For example, mixtures of (Lys-Ala-Lys-Ala)₄ (KAKA)₄ and (Glu-Ala-Glu-Ala)₄ (EAEA)₄ or of (Lys-Ala-Lys-Ala)₄ (KAKA)₄ and (Ala-Asp-Ala-Asp)₄ (ADAD)₄ would be expected to form membranes, but not any of these peptides alone due to lack of complementarity.

Peptides, which are not perfectly complementary or structurally compatible, can be thought of as containing mismatches analogous to mismatched base pairs in the hybridization of nucleic acids. Peptides containing mismatches can form membranes if the disruptive force of the mismatched pair is dominated by the overall stability of the interpeptide interaction.

Functionally, such peptides can also be considered as complementary or structurally compatible. For example, a mismatched amino acid pair may be tolerated if it is surrounded by several perfectly matched pairs on each side.

Each of the peptide sequences disclosed herein may provide for peptides comprising, consisting essentially of, and consisting of the amino acid sequences recited.

The present disclosure provides materials, methods, and kits for solutions, hydrogels, and scaffolds comprising, consisting essentially of, or consisting of the peptides recited herein.

A 1 weight per volume (w/v) percent aqueous (water) solution and a 2.5 w/v percent of (RADA)4 is available as the product PuraMatrix™ peptide hydrogel by 3-D Matrix Co., Ltd.

Certain peptides may contain sequences which are similar to the cell attachment ligand RGD (Arginine-Glycine-Aspartic acid). The suitability of these peptides for supporting in vitro cell growth was tested by introducing a variety of cultured primary and transformed cells to homopolymer sheets of Ala-Glu-Ala-Glu-Ala-Lys-Ala-Lys-Ala-Glu-Ala-Glu-Ala-Lys-Ala-Lys (AEAEAKAKAEAEAKAK (EAK16)), RAD16, RADA16, and heteropolymers of RAD16 and EAK16. The RAD-based peptides may be of particular interest because the similarity of this sequence to RGD. The RAD sequence is a high affinity ligand present in the extracellular matrix protein tenascin and is recognized by integrin receptors. The EAK16 peptide and other peptides disclosed herein were derived from a region of a yeast protein, zuotin.

A list of peptides that may form membranes, hydrogels or scaffolds in homogeneous or heterogeneous mixtures are listed in Table 1.

TABLE 1
Potential hydrogel-forming peptides

| NAME | SEQUENCE (N → C) | SEQ ID NO |
|---|---|---|
| RADA | RADA | SEQ ID NO: 1 |
| IEIK | IEIK | SEQ ID NO: 2 |
| TTTT | TTTT | SEQ ID NO: 3 |
| ATAT | ATAT | SEQ ID NO: 4 |
| TVTV | TVTV | SEQ ID NO: 5 |
| ASAS | ASAS | SEQ ID NO: 6 |
| SSSS | SSSS | SEQ ID NO: 7 |
| VVVTTTT | VVVTTTT | SEQ ID NO: 8 |
| KLDL | KLDL | SEQ ID NO: 9 |
| KLD | KLD | SEQ ID NO: 10 |
| (RADA)₄ | RADARADARADARADA | SEQ ID NO: 11 |
| (IEIK)₃I | IEIKIEIKIEIKI | SEQ ID NO: 12 |
| (IEIK)₄I | IEIKIEIKIEIKIEIKI | SEQ ID NO: 13 |
| (KLDL)₃ | KLDLKLDLKLDL | SEQ ID NO: 14 |
| Peptide A | VRVRVDVDVRVRVDVD | SEQ ID NO: 15 |
| Peptide B | ADADAKAKADADAKAK | SEQ ID NO: 16 |
| (KAKA)₄ | KAKAKAKAKAKAKAKA | SEQ ID NO: 17 |
| (EAEA)₄ | EAEAEAEAEAEAEAEA | SEQ ID NO: 18 |
| (ADAD)₄ | ADADADADADADADAD | SEQ ID NO: 19 |
| EAK16 | AEAEAKAKAEAEAKAK | SEQ ID NO: 20 |
| RAD16 | ARADARADARADARAD | SEQ ID NO: 21 |
| KAKA16 | KAKAKAKAKAKAKAKA | SEQ ID NO: 22 |
| KAKA5 | KAKAK | SEQ ID NO: 23 |
| KAE16 | AKAKAEAEAKAKAEAE | SEQ ID NO: 24 |
| AKE16 | AKAEAKAEAKAEAKAE | SEQ ID NO: 25 |
| EKA16 | EAKAEAKAEAKAEAKA | SEQ ID NO: 26 |
| EAK8 | AEAEAKAK | SEQ ID NO: 27 |
| EAK12 | AEAKAEAEAKAK | SEQ ID NO: 28 |
| KEA16 | KAEAKAEAKAEAKAEA | SEQ ID NO: 29 |
| AEK16 | AEAKAEAKAEAKAEAK | SEQ ID NO: 30 |
| ARD8 | ARARADAD | SEQ ID NO: 31 |
| DAR16 | ADADARARADADARAR | SEQ ID NO: 32 |
| RAD16 | ARADARADARADARAD | SEQ ID NO: 33 |
| DRA16 | DARADARADARADARA | SEQ ID NO: 34 |
| ADR16 | ADARADARADARADAR | SEQ ID NO: 35 |
| ARA16 | ARARADADARARADAD | SEQ ID NO: 36 |
| ARDAKE16 | ARADAKAEARADAKAE | SEQ ID NO: 37 |
| AKEW16 | AKAEARADAKAEARAD | SEQ ID NO: 38 |
| ARKADE16 | ARAKADAEARAKADAE | SEQ ID NO: 39 |
| AKRAED16 | AKARAEADAKARADAE | SEQ ID NO: 40 |
| AQ16 | AQAQAQAQAQAQAQAQ | SEQ ID NO: 41 |
| VQ16 | VQVQVQVQVQVQVQVQ | SEQ ID NO: 42 |
| YQ16 | YQYQYQYQYQYQYQYQ | SEQ ID NO: 43 |
| HQ16 | HQHQHQHQHQHQHQHQ | SEQ ID NO: 44 |
| AN16 | ANANANANANANANAN | SEQ ID NO: 45 |
| VN16 | VNVNVNVNVNVNVNVN | SEQ ID NO: 46 |
| YN16 | YNYNYNYNYNYNYNYN | SEQ ID NO: 47 |
| HN16 | HNHNHNHNHNHNHNHN | SEQ ID NO: 48 |
| ANQ16 | ANAQANAQANAQANAQ | SEQ ID NO: 49 |
| AQN16 | AQANAQANAQANAQAN | SEQ ID NO: 50 |
| VNQ16 | VNVQVNVQVNVQVNVQ | SEQ ID NO: 51 |
| VQK16 | VQVNVQVNVQVNVQVN | SEQ ID NO: 52 |
| YNQ16 | YNYQYNYQYNYQYNYQ | SEQ ID NO: 53 |
| YQN16 | YQYNYQYNYQYNYQYN | SEQ ID NO: 54 |
| HNQ16 | HNHQHNHQHNHQHNHQ | SEQ ID NO: 55 |
| HQN16 | HQHNHQHNHQHNHQHN | SEQ ID NO: 56 |
| AKQD18 | AKAQADAKAQADAKAQAD | SEQ ID NO: 57 |
| VKQ18 | VKVQVDVKVQVDVKVQVD | SEQ ID NO: 58 |
| YKQ18 | YKYQYDYKYQYDYKYQYD | SEQ ID NO: 59 |
| HKQ18 | HKHQHDHKHQHDHKHQHD | SEQ ID NO: 60 |
| RAD | RAD | SEQ ID NO: 61 |
| AAAAAAK | AAAAAAK | SEQ ID NO: 62 |
| AAAAAAD | AAAAAAD | SEQ ID NO: 63 |
| TTTTTTT | TTTTTTT | SEQ ID NO: 64 |
| ATATATAT | ATATATAT | SEQ ID NO: 65 |
| TVTVTVTV | TVTVTVTV | SEQ ID NO: 66 |
| ASASASAS | ASASASAS | SEQ ID NO: 67 |
| SSSSSSS | SSSSSSS | SEQ ID NO: 68 |
| (RADA)₅₀ | RADARADARADARADARADARADARADARA | SEQ ID NO: 69 |
| | DARADARADARADARADARADARADARAD | |
| | ARADARADARADARADARADARADARADAR | |
| | ADARADARADARADARADARADARADARA | |
| | DARADARADARADARADARADARADARAD | |
| | ARADARADARADA | |
| | RADARADARADARADARADARADARADARA | |
| | DARADARADA | |
| (IEIK)₅₀ | IEIKIEIKIEIKIEIKIEIKIEIKIEIKIEIKIEIKIEIKIE | SEQ ID NO: 70 |
| | IKIEIKIEIKIEIKIEIKIEIKIEIKIEIKIEIKIEIKIEIK | |
| | IEIKIEIKIEIKIEIKIEIKIEIKIEIKIEIKIEIKIEIKIE | |
| | IKIEIKIEIKIEIKIEIKIEIKIEIKIEIKIEIKIEIKIEIK | |
| | IEIKIEIKIEIKIEIKIEIKIEIKIEIKIEIK | |
| (IEIK)₅₀I | IEIKIEIKIEIKIEIKIEIKIEIKIEIKIEIKIEIKIEIKIE | SEQ ID NO: 71 |
| | IKIEIKIEIKIEIKIEIKIEIKIEIKIEIKIEIKIEIKIEIK | |
| | IEIKIEIKIEIKIEIKIEIKIEIKIEIKIEIKIEIKIEIKIE | |
| | IKIEIKIEIKIEIKIEIKIEIKIEIKIEIKIEIKIEIKIEIK | |
| | IEIKIEIKIEIKIEIKIEIKIEIKIEIKIEIKI | |
| (KLDL)₅₀ | KLDLKLDLKLDLKLDLKLDLKLDLKLDLKLD | SEQ ID NO: 72 |
| | LKLDLKLDLKLDLKLDLKLDLKLDLKLDLKL | |
| | DLKLDLKLDLKLDLKLDLKLDLKLDLKLDLK | |
| | LDLKLDLKLDLKLDLKLDLKLDLKLDLKLDL | |
| | KLDLKLDLKLDLKLDLKLDLKLDLKLDLKLD | |
| | LKLDLKLDLKLDLKLDLKLDLKLDLKLDLKL | |
| | DLKLDLKLDLKLDL | |
| (KLD)₅₀ | KLDKLDKLDKLDKLDKLDKLDKLDKLDKLD | SEQ ID NO: 73 |
| | KLDKLDKLDKLDKLDKLDKLDKLDKLDKLD | |
| | KLDKLDKLDKLDKLDKLDKLDKLDKLDKLD | |
| | KLDKLDKLDKLDKLDKLDKLDKLDKLDKLD | |
| | KLDKLDKLDKLDKLDKLDKLDKLDKLDKLD | |
| (KLDL)₂ | KLDLKLDL | SEQ ID NO: 74 |
| (KLDL)₃ | KLDLKLDLKLDL | SEQ ID NO: 75 |
| (AGAG)₄ | AGAGAGAGAGAGAGAG | SEQ ID NO: 76 |
| (LALA)₄ | LALALALALALALALA | SEQ ID NO: 77 |
| LALAL | LALAL | SEQ ID NO: 78 |
| (ALALAGAG)₂ | ALALAGAGALALAGAG | SEQ ID NO: 79 |
| (ALAG)₄ | ALAGALAGALAGALAG | SEQ ID NO: 80 |
| (GALA)₄ | GALAGALAGALAGALA | SEQ ID NO: 81 |
| AGAGALAL | AGAGALAL | SEQ ID NO: 82 |
| AGALAGAGAL | AGALAGAGALAL | SEQ ID NO: 83 |
| AL | | |
| (LAGA)₄ | LAGALAGALAGALAGA | SEQ ID NO: 84 |
| (AGAL)₄ | AGALAGALAGALAGAL | SEQ ID NO: 85 |

Without wishing to be bound by any particular theory, it is believed that the self-assembly of the peptides may be attributable to hydrogen bonding and hydrophobic bonding between the peptide molecules by the amino acids composing the peptides.

As used herein, an “effective amount” or a “therapeutically effective amount” refers to an amount of a peptide, peptide solution or hydrogel effective to promote or provide a pancreatic fistula occlusion in a subject. In certain embodiments, such an “effective amount” or “therapeutically effective amount” may refer to an amount of a peptide, peptide solution or hydrogel which is effective, upon single or multiple administration (application or injection) to a subject, in treating, or in curing, alleviating, relieving or improving a subject with a disorder beyond that expected in the absence of such treatment. This may include a particular concentration or range of concentrations of peptide in the peptide solution or hydrogel and additionally, or in the alternative, a particular volume or range of volumes of the peptide solution or hydrogel. The method of facilitating may comprise providing instructions to prepare at least one of the effective amount and the effective concentration.

The self-assembling peptides of the present disclosure, such as RADA16, may be peptide sequences that lack a distinct physiologically or biologically active motif or sequence, and therefore may not impair intrinsic cell function. Physiologically active motifs may control numerous intracellular phenomena such as transcription, and the presence of physiologically active motifs may lead to phosphorylation of intracytoplasmic or cell surface proteins by enzymes that recognize the motifs. When a physiologically active motif is present in a peptide tissue occluding agent, transcription of proteins with various functions may be activated or suppressed. The self-assembling peptides, of the present disclosure may lack such physiologically active motifs and therefore do not carry this risk.

The optimal lengths for membrane formation may vary with the amino acid composition. A stabilization factor contemplated by the peptides of the present disclosure is that complementary peptides maintain a constant distance between the peptide backbones. Peptides which can maintain a constant distance upon pairing are referred to herein as structurally compatible. The interpeptide distance can be calculated for each ionized or hydrogen bonding pair by taking the sum of the number of unbranched atoms on the side-chains of each amino acid in the pair. For example, lysine has 5 and glutamic acid has 4 unbranched atoms on its side-chains, respectively.

The dosage, for example, volume or concentration, administered (for example, applied or injected) may vary depending upon the form of the peptide (for example, in a peptide solution, hydrogel, or in a dried form, such as a lyophilized form) and the route of administration utilized. The exact formulation, route of administration, volume, and concentration can be chosen in view of the subject's condition and in view of the particular target area or location that the peptide solution, hydrogel, or other form of peptide will be administered. Lower or higher doses than those recited herein may be used or required. Specific dosage and treatment regimens for any particular subject may depend upon a variety of factors, which may include the specific peptide or peptides employed, the dimension of the area that is being treated, the desired thickness of the resulting hydrogel that may be positioned in the desired target area, and the length of time of treatment. Other factors that may affect the specific dosage and treatment regimens include age, body weight, general health status, sex, time of administration, rate of degradation, the severity and course of the disease, condition or symptoms, and the judgment of the treating physician. In certain embodiments, the peptide solution may be administered in a single dose. In other embodiments, the peptide solution may be administered in more than one dose, or multiple doses. The peptide solution may be administered in at least two doses.

An effective amount and an effective concentration of the peptide solution may be selected to at least partially promote or provide an occlusion at or near a pancreatic fistula. In some embodiments, at least one of the effective amount and the effective concentration may be based in part on a dimension or diameter of the target area. In other embodiments, at least one of the effective amount and the effective concentration is based in part on the flow rate of one or more fluids at or near the target area. In still other embodiments, at least one of the effective amount and the effective concentration may be based in part on a dimension or diameter of a site of fluid leakage of a pancreatic fistula.

In yet other embodiments, at least one of the effective amount and the effective concentration may be based in part on at least one of a dimension or diameter of the target area, and the flow rate of one or more fluids at or near the target area, and a dimension or diameter of a site of fluid leakage of a pancreatic fistula.

The effective amount may include volumes of from about 0.1 milliliters (mL) to about 100 mL of a peptide solution. The effective amount may include volumes of from about 0.1 mL to about 10 mL of a peptide solution. In certain embodiments, the effective amount may be about 0.5 mL. In other embodiments, the effective amount may be about 1.0 mL. In yet other embodiments, the effective amount may be about 1.5 mL. In still yet other embodiments, the effective amount may be about 2.0 mL. In some other embodiments, the effective amount may be about 3.0 mL.

In certain embodiments, the effective amount may be approximately 0.1 mL per 1 cm² to approximately 5 mL per 1 cm² of target area. In certain embodiments, the effective amount may be approximately 1 mL per 1 cm² of target area. This effective amount may be used related to a concentration, such as a 2.5 weight per volume percent of a peptide solution of the present disclosure.

The effective concentration may be, as described herein, an amount that may provide for occlusion of a pancreatic fistula. Various properties at or near the target site may contribute to the selection or determination of the effective concentration including at least one of a dimension or diameter of the target area, and the flow rate of one or more fluids at or near the target area.

The effective concentration may include peptide concentrations in the solution in a range of about 0.1 weight per volume (w/v) percent to about 10 w/v percent. The effective concentration may include peptide concentrations in the solution in a range of about 0.1 w/v percent to about 3.5 w/v percent. In certain embodiments, the effective concentration may be about 1 w/v percent. In other embodiments, the effective concentration may be about 2.5 w/v percent. In yet other embodiments, the effective concentration may be about 3.0 w/v percent.

In certain embodiments, a peptide solution having a higher concentration of peptide may provide for a more effective hydrogel that has the ability to stay in place and provide effective occlusion. For purposes of delivering the peptide solution, higher concentrations of peptide solutions may become too viscous to allow for effective and selective administration of the solution. It is possible that if a high enough concentration is not selected, the hydrogel may not be effective in the target area for the desired period of time.

The effective concentration may be selected to provide for a solution that may be administered by injection or other means using a particular diameter or gauge catheter or needle.

Methods of the disclosure contemplate single as well as multiple administrations of a therapeutically effective amount of the peptides, compositions, peptide solutions, membranes, filaments, and hydrogels as described herein. Peptides as described herein may be administered at regular intervals, depending on the nature, severity and extent of the subject's condition. In some embodiments, a peptide, composition, peptide solution, membrane, filament, or hydrogel may be administered in a single administration. In some embodiments, a peptide, composition, peptide solution, or hydrogel described herein is administered in multiple administrations. In some embodiments, a therapeutically effective amount of a peptide, composition, peptide solution, membrane, filament, or hydrogel may be administered periodically at regular intervals. The regular intervals selected may be based on any one or more of the initial peptide concentration of the solution administered, the amount administered, and the degradation rate of the hydrogel formed. For example, after an initial administration, a follow-on administration may occur after, for example, one week, two weeks, four weeks, six weeks, or eight weeks. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject. The pre-determined intervals may be the same for each follow-on administration, or they may be different. In some embodiments, a peptide, peptide solution, or hydrogel may be administered chronically at pre-determined intervals to maintain at least a partial occlusion of pancreatic fistula in a subject over the life of the subject. The pre-determined intervals may be the same for each follow-on administration, or they may be different. This may be dependent on whether the hydrogel formed from the previous administration is partially or totally disrupted or degraded. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject.

The peptides can be chemically synthesized or they can be purified from natural and recombinant sources. Using chemically synthesized peptides may allow the peptide solutions to be deficient in unidentified components such as unidentified components derived from the extracellular matrix of another animal. This property therefore may eliminate concerns of infection, including risk of viral infection compared to conventional tissue-derived biomaterials. This may eliminate concerns of infection including infections such as bovine spongiform encephalopathy (BSE), making the peptide highly safe for medical use.

The initial concentration of the peptide may be a factor in the size and thickness of the membrane, hydrogel, or scaffold formed. In general, the higher the peptide concentration, the higher the extent of membrane or hydrogel formation. Hydrogels, or scaffolds formed at higher initial peptide concentrations (about 10 mg/ml) (about 1.0 w/v percent) may be thicker and thus, likely to be stronger.

Formation of the, membranes, hydrogels, or scaffolds may be very fast, on the order of a few minutes. The formation of the membranes or hydrogels may be irreversible. In certain embodiments, the formation may be reversible, and in other embodiments, the formation may be irreversible. The hydrogel may form instantaneously upon administration to a target area. The formation of the hydrogel may occur within about one to two minutes of administration. In other examples, the formation of the hydrogel may occur within about three to four minutes of administration. In certain embodiments the time it takes to form the hydrogel may be based at least in part on one or more of the concentration of the peptide solution, the volume of peptide solution applied, and the conditions at the area of application or injection (for example, the concentration of monovalent metal cations at the area of application, the pH of the area, and the presence of one or more fluids at or near the area). The process may be unaffected by pH of less than or equal to 12, and by temperature. The membranes or hydrogels may form at temperatures in the range of 1 to 99 degrees Celsius.

The hydrogels may remain in position at the target area for a period of time sufficient to provide a desired effect using the methods and kits of the present disclosure. The desired effect using the methods and kits of the present disclosure may be to treat areas or to assist in healing of areas in which a surgical procedure at or near the site of a surgery was performed or the site of a pancreatic fistula. For example, the desired effect using the methods and kits of the present disclosure may be to treat areas or to assist in healing of areas in which an endoscopic surgery is performed.

The period of time that the membranes or hydrogels may remain at the desired area may be for about 10 minutes. In certain examples, it may remain at the desired area for about 35 minutes. In certain further examples, it may remain at the desired area for one or more days, up to one or more weeks. In other examples, it may remain at the desired area for up to 30 days, or more. It may remain at the desired area indefinitely. In other examples, it may remain at the desired area for a longer period of time, until it is naturally degraded or intentionally removed. If the hydrogel naturally degrades over a period of time, subsequent application or injection of the hydrogel to the same or different location may be performed.

In certain embodiments, the self-assembling peptide may be prepared with one or more components that may provide for enhanced effectiveness of the self-assembling peptide or may provide another action, treatment, therapy, or otherwise interact with one or more components of the subject. For example, additional peptides comprising one or more biologically or physiologically active amino acid sequences or motifs may be included as one of the components along with the self-assembling peptide. Other components may include biologically active compounds such as a drug or other treatment that may provide some benefit to the subject. For example, a cancer treating drug or anticancer drug may be administered with the self-assembling peptide, or may be administered separately.

The peptide, peptide solution, or hydrogel may comprise small molecular drugs to treat the subject or to prevent hemolysis, inflammation, and infection. The small molecular drugs may be selected from the group consisting of glucose, saccharose, purified saccharose, lactose, maltose, trehalose, destran, iodine, lysozyme chloride, dimethylisoprpylazulene, tretinoin tocoferil, povidone iodine, alprostadil alfadex, anise alcohol, isoamyl salicylate, α,α-dimethylphenylethyl alcohol, bacdanol, helional, sulfazin silver, bucladesine sodium, alprostadil alfadex, gentamycin sulfate, tetracycline hydrochloride, sodium fusidate, mupirocin calcium hydrate and isoamyl benzoate. Other small molecular drugs may be contemplated. Protein-based drugs may be included as a component to be administered, and may include erythropoietin, tissue type plasminogen activator, synthetic hemoglobin and insulin.

A component may be included to protect the peptide solution against rapid or immediate formation into a hydrogel. This may include an encapsulated delivery system that may degrade over time to allow a controlled time release of the peptide solution into the target area to form the hydrogel over a desired, predetermined period of time. Biodegradable, biocompatible polymers may be used, such as ethylene vinyl acetate, polyanhydrides, polyglycolic acid, collagen, polyorthoesters, and polylactic acid.

A sugar may be added to the self-assembling peptide solution to improve the osmotic pressure of the solution from hypotonicity to isotonicity without reducing the tissue occluding effect, thereby allowing the biological safety to be increased. In certain examples, the sugar may be sucrose or glucose.

Any of the components described herein may be included in the peptide solution or may be administered separate from the peptide solution. Additionally, any of the methods and methods of facilitating provided herein may be performed by one or more parties.

A peptide, peptide solution, or hydrogel of the disclosure may be provided in a kit. Instructions for administering the solution to a target area of a subject may also be provided in the kit. The peptide solution may comprise a self-assembling peptide comprising at least about 7 amino acids in an effective amount and in an effective concentration to form a hydrogel to at least partially promote or provide occlusion of a pancreatic fistula. The peptide solution may comprise a self-assembling peptide comprising between about 7 amino acids to about 32 amino acids in an effective amount and in an effective concentration to form a hydrogel to at least partially promote or provide occlusion of a pancreatic fistula. The instructions for administering the solution may comprise methods for administering the peptide, peptide solution, or hydrogel provided herein, for example, by a route of administration described herein, at a dose, volume or concentration, or administration schedule. The peptide may be amphiphilic and at least a portion of the peptide may alternate between a hydrophobic amino acid and a hydrophilic amino acid.

The kit may also comprise informational material. The informational material may be descriptive, instructional, marketing or other material that relates to the methods described herein. In one embodiment, the informational material may include information about production of the peptide, peptide solution, or hydrogel disclosed herein, physical properties of the peptide, composition, peptide solution or hydrogel, concentration, volume, size, dimensions, date of expiration, and batch or production site.

The kit may also optionally include a device or materials to allow for administration of the peptide or peptide solution to the desired area. For example, a syringe, pipette, catheter, or other needle-based device may be included in the kit. Additionally, or alternatively, the kit may include a guidewire, endoscope, or other accompanying equipment to provide selective administration of the peptide solution to the target area.

The kit may comprise in addition to or in the alternative, other components or ingredients, such as components that may aid in positioning of the peptide solution, hydrogel or scaffold. Instructions may be provided in the kit to combine a sufficient quantity or volume of the peptide solution with a sucrose solution, that may or may not be provided with the kit. Instructions may be provided for diluting the peptide solution to administer an effective concentration of the solution to the target area. The instruction may describe diluting the peptide solution with a diluent or solvent. The diluent or solvent may be water. Instructions may further be provided for determining at least one of the effective concentration of the solution and the effective amount of the solution to the target area. This may be based on various parameters discussed herein, and may include the diameter of the lesion or site of pancreatic fistula or wound at the target area.

Other components or ingredients may be included in the kit, in the same or different compositions or containers than the peptide, peptide solutions, or hydrogel. The one or more components that may include components that may provide for enhanced effectiveness of the self-assembling peptide or may provide another action, treatment, therapy, or otherwise interact with one or more components of the subject. For example, additional peptides comprising one or more biologically or physiologically active sequences or motifs may be included as one of the components along with the self-assembling peptide. Other components may include biologically active compounds such as a drug or other treatment that may provide some benefit to the subject. For example, a cancer treating drug or anticancer drug may be administered with the self-assembling peptide, or may be administered separately. The peptide, peptide solution, or hydrogel may comprise small molecular drugs to treat the subject or to prevent hemolysis, inflammation, and infection, as disclosed herein. A sugar solution such as a sucrose solution may be provided with the kit. The sucrose solution may be a 20% sucrose solution.

Other components which are disclosed herein may also be included in the kit.

In some embodiments, a component of the kit is stored in a sealed vial, for example, with a rubber or silicone closure (for example, a polybutadiene or polyisoprene closure). In some embodiments, a component of the kit is stored under inert conditions (for example, under nitrogen or another inert gas such as argon). In some embodiments, a component of the kit is stored under anhydrous conditions (for example, with a desiccant). In some embodiments, a component of the kit is stored in a light blocking container such as an amber vial.

As part of the kit or separate from a kit, syringes or pipettes may be pre-filled with a peptide, peptide solution, or hydrogel as disclosed herein. Methods to instruct a user to supply a self-assembling peptide solution to a syringe or pipette, with or without the use of other devices, and administering it to the target area through the syringe or pipette, with or without the use of other devices, is provided. Other devices may include, for example, a catheter with or without a guidewire.

In some embodiments of the disclosure, the self-assembling peptides may be used as a coating on a device or an instrument such as a stent or catheter, to suppress body fluid leakage. The self-assembling peptides may also be incorporated or secured to a support, such as gauze or a bandage, or a lining, that may provide a therapeutic effect to a subject, or that may be applied within a target area. The self-assembling peptides may also be soaked into a sponge for use.

The membranes may also be useful for culturing cell monolayers. Cells prefer to adhere to non-uniform, charged surfaces. The charged residues and conformation of the proteinaceous membranes promote cell adhesion and migration. The addition of growth factors, such as fibroblast growth factor, to the peptide membrane may further improve attachment, cell growth and neurite outgrowth.

Although the peptide solution may be liquid at acidic pH, the peptide may undergo self-organization or self-assembly upon adjustment of a pH level of the solution to a neutral or physiological pH level. The solution may be aqueous or non-aqueous.

The above descriptions are illustrative and not restrictive. Many variations of the technology will become apparent to those of skill in the art upon review of this disclosure. The scope of the technology should, therefore, be determined not with reference to the embodiments described above, but instead should be determined with reference to the appended claims along with their full scope of equivalents.

1. A method for occluding a pancreatic fistula, the method comprising administering an effective amount of a self-assembling peptide solution to a pancreatic fistula, wherein the self-assembling peptide is between about 7 amino acids and 32 amino acids in length and the self-assembling peptide solution forms a hydrogel under physiological conditions, thereby occluding pancreatic fistula.

2. The method of claim 1, wherein the self-assembling peptide comprises about 12 to about 16 amino acids that alternate between hydrophobic and a hydrophilic amino acids.

3. The method of claim 1, wherein the self-assembling peptide comprises a sequence selected from RADA, IEIK, TTTT, ATAT, TVTV, ASAS, SSSS, VVVTTTT, and a combination thereof.

4. The method of claim 1, wherein the self-assembling peptide comprises a sequence selected from (RADA)₄, (IEIK)₃I, and (KLDL)₃.

5. The method of claim 1, wherein the self-assembling peptide is about 0.1 to about 10 w/v % of the solution or about 0.1 to about 3.5 w/v % of the solution.

6. The method of claim 1, wherein the self-assembling peptide is about 1, about 2.5, or about 3 w/v % of the solution.

7. The method of claim 1, wherein the effective amount is approximately 0.1 mL per 1 cm² to approximately 5 mL per 1 cm² of target area.

8. The method of claim 1, wherein the effective amount is approximately 1 mL per 1 cm² of target area.

9. The method of claim 1, wherein the hydrogel is formed before administering the self-assembling peptide solution to the pancreatic fistula.

10. The method of claim 1, wherein the hydrogel is formed after administering the self-assembling peptide solution to the pancreatic fistula.

11. The method of claim 1, wherein the solution further comprises a biologically active agent.

12. The method of claim 1, wherein the solution is substantially free of cells and/or drugs.

13. The method of claim 1, wherein the self-assembling peptide solution is administered in vivo.

14. The method of claim 1, wherein the pancreatic fistula is a human pancreatic fistula.

15.-28. (canceled)


SEQUENCE LISTING (85 sequences)

SEQ ID NO: 1 - protein, 4 aa, Artificial Sequence
  Synthetic peptide
RADA

SEQ ID NO: 2 - protein, 4 aa, Artificial Sequence
  Synthetic peptide
IEIK

SEQ ID NO: 3 - protein, 4 aa, Artificial Sequence
  Synthetic peptide
TTTT

SEQ ID NO: 4 - protein, 4 aa, Artificial Sequence
  Synthetic peptide
ATAT

SEQ ID NO: 5 - protein, 4 aa, Artificial Sequence
  Synthetic peptide
TVTV

SEQ ID NO: 6 - protein, 4 aa, Artificial Sequence
  Synthetic peptide
ASAS

SEQ ID NO: 7 - protein, 4 aa, Artificial Sequence
  Synthetic Peptide
SSSS

SEQ ID NO: 8 - protein, 7 aa, Artificial Sequence
  Synthetic peptide
VVVTTTT

SEQ ID NO: 9 - protein, 4 aa, Artificial Sequence
  Synthetic peptide
KLDL

SEQ ID NO: 10 - protein, 3 aa, Artificial Sequence
  Synthetic peptide
KLD

SEQ ID NO: 11 - protein, 16 aa, Artificial Sequence
  Synthetic peptide
RADARADARADARADA

SEQ ID NO: 12 - protein, 13 aa, Artificial Sequence
  Synthetic peptide
IEIKIEIKIEIKI

SEQ ID NO: 13 - protein, 17 aa, Artificial Sequence
  Synthetic peptide
IEIKIEIKIEIKIEIKI

SEQ ID NO: 14 - protein, 12 aa, Artificial Sequence
  Synthetic peptide
KLDLKLDLKLDL

SEQ ID NO: 15 - protein, 16 aa, Artificial Sequence
  Synthetic peptide
VRVRVDVDVRVRVDVD

SEQ ID NO: 16 - protein, 16 aa, Artificial Sequence
  Synthetic peptide
ADADAKAKADADAKAK

SEQ ID NO: 17 - protein, 16 aa, Artificial Sequence
  Synthetic peptide
KAKAKAKAKAKAKAKA

SEQ ID NO: 18 - protein, 16 aa, Artificial Sequence
  Synthetic peptide
EAEAEAEAEAEAEAEA

SEQ ID NO: 19 - protein, 16 aa, Artificial Sequence
  Synthetic peptide
ADADADADADADADAD

SEQ ID NO: 20 - protein, 16 aa, Artificial Sequence
  Synthetic peptide
AEAEAKAKAEAEAKAK

SEQ ID NO: 21 - protein, 16 aa, Artificial Sequence
  Synthetic peptide
ARADARADARADARAD

SEQ ID NO: 22 - protein, 16 aa, Artificial Sequence
  Synthetic peptide
KAKAKAKAKAKAKAKA

SEQ ID NO: 23 - protein, 5 aa, Artificial Sequence
  Synthetic peptide
KAKAK

SEQ ID NO: 24 - protein, 16 aa, Artificial Sequence
  Synthetic peptide
AKAKAEAEAKAKAEAE

SEQ ID NO: 25 - protein, 16 aa, Artificial Sequence
  Synthetic peptide
AKAEAKAEAKAEAKAE

SEQ ID NO: 26 - protein, 16 aa, Artificial Sequence
  Synthetic peptide
EAKAEAKAEAKAEAKA

SEQ ID NO: 27 - protein, 8 aa, Artificial Sequence
  Synthetic peptide
AEAEAKAK

SEQ ID NO: 28 - protein, 12 aa, Artificial Sequence
  Synthetic peptide
AEAKAEAEAKAK

SEQ ID NO: 29 - protein, 16 aa, Artificial Sequence
  Synthetic peptide
KAEAKAEAKAEAKAEA

SEQ ID NO: 30 - protein, 16 aa, Artificial Sequence
  Synthetic peptide
AEAKAEAKAEAKAEAK

SEQ ID NO: 31 - protein, 8 aa, Artificial Sequence
  Synthetic peptide
ARARADAD

SEQ ID NO: 32 - protein, 16 aa, Artificial Sequence
  Synthetic peptide
ADADARARADADARAR

SEQ ID NO: 33 - protein, 16 aa, Artificial Sequence
  Synthetic peptide
ARADARADARADARAD

SEQ ID NO: 34 - protein, 16 aa, Artificial Sequence
  Synthetic peptide
DARADARADARADARA

SEQ ID NO: 35 - protein, 16 aa, Artificial Sequence
  Synthetic peptide
ADARADARADARADAR

SEQ ID NO: 36 - protein, 16 aa, Artificial Sequence
  Synthetic peptide
ARARADADARARADAD

SEQ ID NO: 37 - protein, 16 aa, Artificial Sequence
  Synthetic peptide
ARADAKAEARADAKAE

SEQ ID NO: 38 - protein, 16 aa, Artificial Sequence
  Synthetic peptide
AKAEARADAKAEARAD

SEQ ID NO: 39 - protein, 16 aa, Artificial Sequence
  Synthetic peptide
ARAKADAEARAKADAE

SEQ ID NO: 40 - protein, 16 aa, Artificial Sequence
  Synthetic peptide
AKARAEADAKARADAE

SEQ ID NO: 41 - protein, 16 aa, Artificial Sequence
  Synthetic peptide
AQAQAQAQAQAQAQAQ

SEQ ID NO: 42 - protein, 16 aa, Artificial Sequence
  Synthetic peptide
VQVQVQVQVQVQVQVQ

SEQ ID NO: 43 - protein, 16 aa, Artificial Sequence
  Synthetic peptide
YQYQYQYQYQYQYQYQ

SEQ ID NO: 44 - protein, 16 aa, Artificial Sequence
  Synthetic peptide
HQHQHQHQHQHQHQHQ

SEQ ID NO: 45 - protein, 16 aa, Artificial Sequence
  Synthetic peptide
ANANANANANANANAN

SEQ ID NO: 46 - protein, 16 aa, Artificial Sequence
  Synthetic peptide
VNVNVNVNVNVNVNVN

SEQ ID NO: 47 - protein, 16 aa, Artificial Sequence
  Synthetic peptide
YNYNYNYNYNYNYNYN

SEQ ID NO: 48 - protein, 16 aa, Artificial Sequence
  Synthetic peptide
HNHNHNHNHNHNHNHN

SEQ ID NO: 49 - protein, 16 aa, Artificial Sequence
  Synthetic peptide
ANAQANAQANAQANAQ

SEQ ID NO: 50 - protein, 16 aa, Artificial Sequence
  Synthetic peptide
AQANAQANAQANAQAN

SEQ ID NO: 51 - protein, 16 aa, Artificial Sequence
  Synthetic peptide
VNVQVNVQVNVQVNVQ

SEQ ID NO: 52 - protein, 16 aa, Artificial Sequence
  Synthetic peptide
VQVNVQVNVQVNVQVN

SEQ ID NO: 53 - protein, 16 aa, Artificial Sequence
  Synthetic peptide
YNYQYNYQYNYQYNYQ

SEQ ID NO: 54 - protein, 16 aa, Artificial Sequence
  Synthetic peptide
YQYNYQYNYQYNYQYN

SEQ ID NO: 55 - protein, 16 aa, Artificial Sequence
  Synthetic peptide
HNHQHNHQHNHQHNHQ

SEQ ID NO: 56 - protein, 16 aa, Artificial Sequence
  Synthetic peptide
HQHNHQHNHQHNHQHN

SEQ ID NO: 57 - protein, 18 aa, Artificial Sequence
  Synthetic peptide
AKAQADAKAQADAKAQAD

SEQ ID NO: 58 - protein, 18 aa, Artificial Sequence
  Synthetic peptide
VKVQVDVKVQVDVKVQVD

SEQ ID NO: 59 - protein, 18 aa, Artificial Sequence
  Synthetic peptide
YKYQYDYKYQYDYKYQYD

SEQ ID NO: 60 - protein, 18 aa, Artificial Sequence
  Synthetic peptide
HKHQHDHKHQHDHKHQHD

SEQ ID NO: 61 - protein, 3 aa, Artificial Sequence
  Synthetic peptide
RAD

SEQ ID NO: 62 - protein, 7 aa, Artificial Sequence
  Synthetic peptide
AAAAAAK

SEQ ID NO: 63 - protein, 7 aa, Artificial Sequence
  Synthetic peptide
AAAAAAD

SEQ ID NO: 64 - protein, 7 aa, Artificial Sequence
  Synthetic peptide
TTTTTTT

SEQ ID NO: 65 - protein, 8 aa, Artificial Sequence
  Synthetic peptide
ATATATAT

SEQ ID NO: 66 - protein, 8 aa, Artificial Sequence
  Synthetic peptide
TVTVTVTV

SEQ ID NO: 67 - protein, 8 aa, Artificial Sequence
  Synthetic peptide
ASASASAS

SEQ ID NO: 68 - protein, 7 aa, Artificial Sequence
  Synthetic peptide
SSSSSSS

SEQ ID NO: 69 - protein, 200 aa, Artificial Sequence
  Synthetic peptide
  MISC_FEATURE (1)..(200): /note=“This sequence may encompass 2-50 ′Arg Ala Asp Ala′ repeating units”
  MISC_FEATURE (1)..(200): /note=“Variant residues given in the sequence have no preference with respect to those in the annotations for variant positions”
  VARIANT (9)..(200): /replace=“ ”
RADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADA

SEQ ID NO: 70 - protein, 200 aa, Artificial Sequence
  Synthetic peptide
  MISC_FEATURE (1)..(200): /note=“This sequence may encompass 2-50 ′Ile Glu Ile Lys′ repeating units”
  MISC_FEATURE (1)..(200): /note=“Variant residues given in the sequence have no preference with respect to those in the annotations for variant positions”
  VARIANT (9)..(200): /replace=“ ”
IEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIK

SEQ ID NO: 71 - protein, 201 aa, Artificial Sequence
  Synthetic peptide
  MISC_FEATURE (1)..(201): /note=“This sequence may encompass 2-50 ′Ile Glu Ile Lys′ repeating units”
  MISC_FEATURE (1)..(201): /note=“Variant residues given in the sequence have no preference with respect to those in the annotations for variant positions”
  VARIANT (9)..(201): /replace=“ ”
IEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKI

SEQ ID NO: 72 - protein, 200 aa, Artificial Sequence
  Synthetic peptide
  MISC_FEATURE (1)..(200): =note=“This sequence may encompass 2-50 ′Lys Leu Asp Leu′ repeating units”
  MISC_FEATURE (1)..(200): =note=“Variant residues given in the sequence have no preference with respect to those in the annotations for variant positions”
  VARIANT (9)..(200): /replace=“ ”
KLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDL

SEQ ID NO: 73 - protein, 150 aa, Artificial Sequence
  Synthetic peptide
  MISC_FEATURE (1)..(150): /note=“This sequence may encompass 2-50 ′Lys Leu Asp′ repeating units”
  MISC_FEATURE (1)..(150): /note=“Variant residues given in the sequence have no preference with respect to those in the annotations for variant positions”
  VARIANT (7)..(150): /replace=“ ”
KLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLD

SEQ ID NO: 74 - protein, 8 aa, Artificial Sequence
  Synthetic peptide
KLDLKLDL

SEQ ID NO: 75 - protein, 12 aa, Artificial Sequence
  Synthetic peptide
KLDLKLDLKLDL

SEQ ID NO: 76 - protein, 16 aa, Artificial Sequence
  Synthetic peptide
AGAGAGAGAGAGAGAG

SEQ ID NO: 77 - protein, 16 aa, Artificial Sequence
  Synthetic peptide
LALALALALALALALA

SEQ ID NO: 78 - protein, 5 aa, Artificial Sequence
  Synthetic peptide
LALAL

SEQ ID NO: 79 - protein, 16 aa, Artificial Sequence
  Synthetic peptide
ALALAGAGALALAGAG

SEQ ID NO: 80 - protein, 16 aa, Artificial Sequence
  Synthetic peptide
ALAGALAGALAGALAG

SEQ ID NO: 81 - protein, 16 aa, Artificial Sequence
  Synthetic peptide
GALAGALAGALAGALA

SEQ ID NO: 82 - protein, 8 aa, Artificial Sequence
  Synthetic peptide
AGAGALAL

SEQ ID NO: 83 - protein, 12 aa, Artificial Sequence
  Synthetic peptide
AGALAGAGALAL

SEQ ID NO: 84 - protein, 16 aa, Artificial Sequence
  Synthetic peptide
LAGALAGALAGALAGA

SEQ ID NO: 85 - protein, 16 aa, Artificial Sequence
  Synthetic peptide
AGALAGALAGALAGAL